(19) United States
(12) Patent Application Publication
(10) Pub. No.: US 2021/0308880 A1
(43) Pub. Date: Oct. 7, 2021

(54) Self-Disinfecting Robot and Method For Disinfecting A Robot

(71) Applicant: ABB Schweiz AG, Baden (CH)

(72) Inventors: Frederic Tholence, Mölndal (SE); Erik Johansson, Västerås (SE); Orlando Girlanda, Västerås (SE); Santanu Singha, Västerås (SE)

(21) Appl. No.: 17/290,492

(22) PCT Filed: Nov. 7, 2018
(86) PCT No.: PCT/EP2018/080490

§ 371 Date: Apr. 30, 2021

(51) Int. Cl. B25J 19/00; A61L 2/10; A61L 2/24

(52) U.S. Cl. CPC B25J 19/0058; A61L 2/10; A61L 2202/14; A61L 2202/11; A61L 2/24

(57) ABSTRACT

A self-disinfecting robot including an exterior surface. The robot includes at least one light emitting component arranged to illuminate at least a part of the exterior surface with disinfecting light from inside the robot. By projecting disinfecting light from the inside of the robot, one may provide a constant and complete disinfection of the exterior surface (or selected parts of it) e.g. during service or operation, including when the robot is moving around. The solution also allows disinfection of e.g. crevices that would also be difficult to disinfect from the outside. Furthermore, the robot may also, to some extent, treat the surrounding environment and in particular the airborne particles around the robot. The disclosure also relates to a corresponding method for disinfecting a robot, to a computer program and to a computer program product.

### TECHNICAL FIELD

The present disclosure relates to robotics, and in particular to a self-disinfecting robot. The disclosure also relates to a corresponding method for disinfecting a robot, to a computer program and to a computer program product for performing the method.

### BACKGROUND

We are at the beginning of a new era where collaborative and service robots will be part of our everyday lives. Collaborative robots, a.k.a. Cobots, inside factories are already today accomplishing monotonous tasks next to humans. It is also forecasted that robots will be increasingly used in production lines in the Food and Beverage, FnB, industry, pharmaceutical laboratories, farms, hospitals etc. However, the increased proximity between robots and humans (or products consumed by humans) implies strict demands on their hygienic design and anti-bacterial surface properties.

The main purpose of these disinfected surfaces is to prevent growth and spread of harmful microorganisms onto the robot surface and elsewhere in the factory. Today, disinfected surfaces are commonly achieved by washing the robot at certain time intervals using e.g. toxic chemicals which may also harm the equipment.

Another critical aspect for the use of service robots is the mobility. Placing a robot on a moving platform in a possibly contaminated environment might accelerate spread of contaminants, as bacteria may be carried by the robot while moving around. This problem is not solved by the traditional washing.

Consequently, there is a need for improved ways of disinfecting robots. In particular, there is a need for an improved way of disinfecting robots during operation and service of the robot.

### SUMMARY

It is thus an object of the disclosure to alleviate at least some of the drawbacks with the prior art. It is an object to provide an alternative way of preventing the growth and spread of harmful microorganisms on the exterior surface of a robot. In particular it is an object to provide a solution which is gentle to the environment and which does also not harm the robot or the staff in the factory.

According to a first aspect, the disclosure relates to a self-disinfecting robot (herein referred to as simply a robot) comprising an exterior surface. The robot comprises at least one light emitting component arranged to illuminate at least a part of the exterior surface with disinfecting light from inside the robot. By projecting disinfecting light from the inside of the robot, one may provide a constant and complete disinfection of the exterior surface (or selected parts of it) e.g. during service or operation, including when the robot is moving around. The solution also allows disinfection of e.g. crevices that would also be difficult to disinfect from the outside. Furthermore, the robot may also, to some extent, treat the surrounding environment and in particular the airborne particles around the robot.

According to some embodiments, the at least one light emitting component is configured to illuminate the exterior surface from the inside by emitting the disinfecting light from below the exterior surface or from the side along the exterior surface. Hence, dust and contamination on the exterior (or outer) robot surfaces will not prevent the sanitizing process. This also allows the use of the proposed technique in congested environments.

According to some embodiments, the at least one light emitting component comprises an internal light source configured to emit the disinfecting light arranged inside the robot. Thereby, the illumination system itself is protected from external influence. Also, the inner part/volume of the robot will, to some extent, be sanitized as parts of the light will typically illuminate the interior of the robot as well.

According to some embodiments the at least one light emitting component comprises a light guide comprising an aperture, wherein the light guide is arranged to guide the disinfecting light from an external light source, through the light guide and out through the aperture. Hence, an external light guide may be used for several robots. Also, as the light source is external to the robot, it is more easily repaired if broken than if arranged internal to the robot.

According to some embodiments, the robot is designed such that at least a part of the disinfecting light can pass through, at least parts of, the exterior surface. Thereby, a light source positioned under the exterior surface of the robot may be used to disinfect the surface.

According to some embodiments; the at least one light emitting component is arranged to illuminate parts of the exterior surface adjacent to one or more joints and/or interfaces of the robot. The proposed technique offers the possibility of either disinfecting the entire structure or only parts of a robot such as sensitive areas (dynamic and static seals) or grippers.

According to some embodiments, the exterior surface is at least partly defined by a casing and wherein the light emitting component is configured to emit the disinfecting light from a position inside the casing or from a position integrated in the casing.

According to some embodiments, the robot comprises an optical component configured to spread the disinfecting light along the exterior surface, to guide the light to the surface and/or to focus the light on parts of the exterior surface.

According to some embodiments, the optical component comprises a light guide, a light diffusing material and/or a lens. Thereby, the entire exterior surface (or parts of it) may be properly illuminated using a limited number of light emitting components. Also, for severe applications the optical component may provide local illumination of sensitive parts such as sealings (dynamic, static), grippers etc.

According to some embodiments, a part of the exterior surface is defined by a sealing device and wherein the at least one light emitting component is at least partly integrated in the sealing device or wherein the sealing device is transparent to the disinfecting light and wherein light emitting component is arranged to illuminate the exterior surface from the side of the sealing device along the exterior surface or from below the sealing device. Thereby, for example joints, where bacteria and dirt may otherwise enter the robot, may be sanitised.

According to some embodiments, the robot comprises a control unit configured to control the at least one light emitting component and/or the optical component to illuminate at least a part of the exterior surface with disinfecting light from the inside of the robot. Hence, the illumination may be controlled and adapted to the application.

According to some embodiments, the disinfecting light has a wavelength of 200-500 nm. According to some embodiments, the exterior surface is, at least partly covered by a contaminant-activated photocatalytic polymer or glass. Then the wavelength of the disinfecting light would be irrelevant.

According to some embodiments, the robot is mounted on a mobile platform and the at least one light emitting component is configured to emit disinfecting light on the surface, on which the mobile platform is disposed, and/or on one or more transportation means of the mobile platform, to enable sanitization during movement of the mobile platform. Thereby, the robot structure itself could be used as a (contactless) cleaning tool for the surroundings to disinfect airborne particles, the product itself, surrounding appliances, etc.

According to a second aspect, the disclosure relates to a method for disinfecting a robot comprising an exterior surface. The method comprises illuminating at least a part of the exterior surface with disinfecting light from an interior of the robot.

According to some embodiments, wherein the illuminating is continuous, pulsed or triggered based on one or more pre-determined criteria. Thereby, the disinfecting may be adapted depending on the degree of contamination or activated only at given times of day etc. to provide efficient sanitising.

According to some embodiments, the method comprises varying an intensity and/or wavelength of the disinfecting light based on one or more pre-determined criteria. Thereby, the disinfecting may be adapted based on material, presence of people etc. in order not to cause any harm to environment or people, while still providing efficient sanitising.

### BRIEF DESCRIPTION OF THE DRAWINGS

FIG. 1 illustrates a self-disinfecting robot configured to illuminate a major portion of its exterior surface with disinfecting light.

FIGS. 2a to 2c illustrate three example embodiments of light emitting components.

FIG. 3 illustrates an example of a structure of a robot part.

FIGS. 4a to 4c illustrate a first example embodiment of the proposed technique implemented in a robot part comprising the structure of FIG. 3.

FIGS. 5a to 5e illustrate a second example embodiment of the proposed technique implemented in a robot part comprising the structure of FIG. 3.

FIGS. 6a to 6c illustrate a self-disinfecting robot, where only the joints are disinfected.

FIG. 7 illustrates the method for disinfecting a robot according to the second aspect.

### DETAILED DESCRIPTION

An alternative method to conventional washing would be to use Ultraviolet, UV, light for disinfection. UV light is already today used for a large variety of applications in the food industry. With high-performance UV light sources and equipment, it has been demonstrated that water, air and surfaces can be reliably disinfected, cleaned and treated. The use of chemicals can thereby be reduced or even avoided in an economical and environmentally friendly way. However, UV radiation might damage polymeric parts of the equipment and also injure humans. Hence, due to health risks, one should generally avoid exposing humans to any more UV light than necessary.

An alternative to UV light is blue light, which has almost as good disinfecting effect. Blue light technology is used e.g. in clean rooms (e.g. (manufactured by the company Led Tailor innovation—www.LEDtailor.fi) and has demonstrated a significant decrease of microbial particles in air (up to 98% for small particles).

However, for proper disinfection of a surface, the entire surface must be illuminated. UV and blue light lamps used for disinfection are typically mounted over exposed areas, such as in the ceiling or over a lab bench. It may be difficult to achieve a sufficient degree of disinfection of a robot using UV light, in particular while operating the robot, as the robot's exposed surface may be shadowed by objects, dust and contamination. It may also be difficult to make the light reach into crevices etc.

This disclosure proposes a robot with built in disinfection arrangement that emits disinfecting light to the robot's exposed surfaces. According to one embodiment of the invention the disinfecting light is emitted from the inside of the robot, instead of from above. One may illuminate only the most exposed areas of the robot, this would typically be the gripper or foundation, or the entire robot may be illuminated.

The proposed technique will now be described in further detail with reference to the figures.

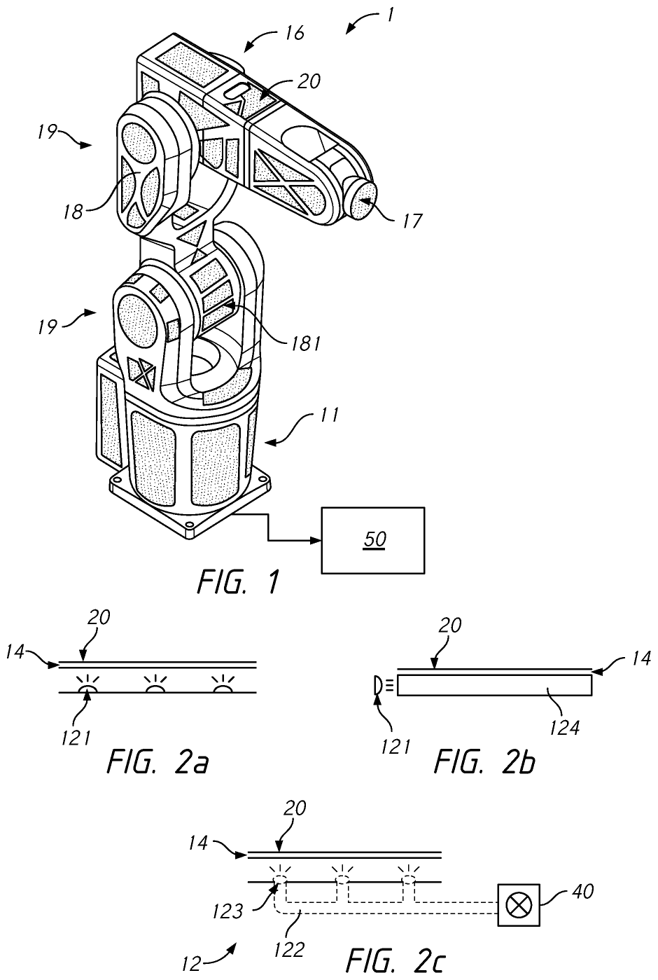

FIG. 1 illustrates an example embodiment of a self-disinfecting robot 1 configured to illuminate a major portion (illustrated as dotted fields) of its exterior surface 20 with disinfecting light from the inside of the robot 1. In FIG. 1, the robot 1 is an industrial robot comprising one robot arm 16 jointly connected to a foot 11. However, the proposed technique may be used on any industrial robot 1. In some embodiments the robot is mounted on a mobile platform. The mobile platform may comprise transportation means, e.g. wheels or a band, such that the robot 1 may move around during operation. In operation, a robot tool, such as a gripper, is typically attached to the interface 17 at the outer part of the robot arm 16. The robot arm 16 comprises one or more robot arm sections connected to each other via joints 19 and motion mechanisms adapted to set the robot arm 16 in motion. Each motion mechanism typically comprises a motor unit and a brake unit (not shown).

The robot 1 comprises an exterior surface 20. The exterior surface 20 is a surface of the robot that is in direct contact with the exterior of the robot 1. At least one light emitting component 12 (FIG. 2a to FIG. 2c) is arranged to illuminate at least a part of the exterior surface 20 with disinfecting light from inside the robot 1. For example, the light emitting component 12 is arranged inside the robot 1 and the robot, or a part of the robot 1, is covered with an outer shell (e.g. a casing or housing) that is transparent to the disinfecting lights. It should be noted that the proposed technique may be implemented in the robot 1 or in individual parts of the robot 1, such as in a robot arm section, in a mobile robot platform or in a robot tool, e.g. a gripper.

In some embodiments, the disinfecting light has a wavelength of 200-500 nm. In some embodiments the disinfecting light is UV light i.e. 380-500 nm. However, as mentioned above UV light is not suitable in all situations. Instead of UV light, one may use intensive blue light (not in the UV spectrum) i.e. 200-380 nm (e.g. 365 nm), to obtain, at least nearly, as good effect as UV light. With blue light, there disinfecting light may be directed towards people, without the health risks associated with UV light.

Alternatively, contaminant-activated visible light photocatalysis may be used. This implies that a substance (more specifically a contaminant-activated photocatalytic polymer) is disposed on the exterior surface 20 and that bacteria that is in contact with the substance activates its own photocatalytic degradation under visible light. Hence, the disinfecting light may be visible light (an ordinary lamp) if the exterior surface 20 is at least partly covered by a contaminant-activated photocatalytic polymer or glass.

In some embodiments the at least one light emitting component 12 is configured to illuminate the exterior surface 20 from the inside by emitting the disinfecting light from below the exterior surface 20. For example, light emitting components 12, here comprising light sources 121 in the form of light diodes, are arranged inside the robot 1, under (or below) the exterior surface 20 to emit the disinfecting light through the exterior surface 20, as illustrated in FIG. 2a. The disinfecting light emitted by the light sources 121 then passes through the exterior surface 20 i.e. through a material (e.g. a food compatible polymer) that is transparent to the disinfecting light. In other words, in some embodiments the robot 1 is designed such that at least a part of the disinfecting light can pass through, at least parts of, the exterior surface 20. For example, parts of the exterior surface that are positioned at areas exposed to contamination may be transparent to the disinfecting light.

In some embodiments the at least one light emitting component 12 is configured to illuminate the exterior surface 20 from the side, i.e. laterally, along the exterior surface 20, as illustrated in FIG. 2b. Then, an optical component in the form of a light guide surface 124 may be used to distribute the light to selected parts of the exterior surface 20.

The actual light source emitting the disinfecting light may alternatively be arranged outside the robot 1, as illustrated in FIG. 2c. Then, the at least one light emitting component 12 may comprise a light guide 122 comprising a plurality of apertures 123. The light guide 122 is arranged to guide the disinfecting light from an external light source 40, through the light guide 122 and out through the apertures 123. The external light source 40 may thus be positioned outside or external to the robot 1.

In some embodiments, the robot 1 comprises an optical component 14, 124 configured to spread the disinfecting light along the exterior surface 20, to guide the light to the surface and/or to focus the light on parts of the exterior surface 20. For example, the optical component may be a light diffusing material 14, e.g. a light diffusing film, that may be arranged (e.g. glues or sprayed) at the exterior surface 20 to provide an even illumination, see FIG. 2a, 2b or 2c. Alternatively, a lens may be used to focus the disinfecting lights on exposed parts of the exterior surface 20.

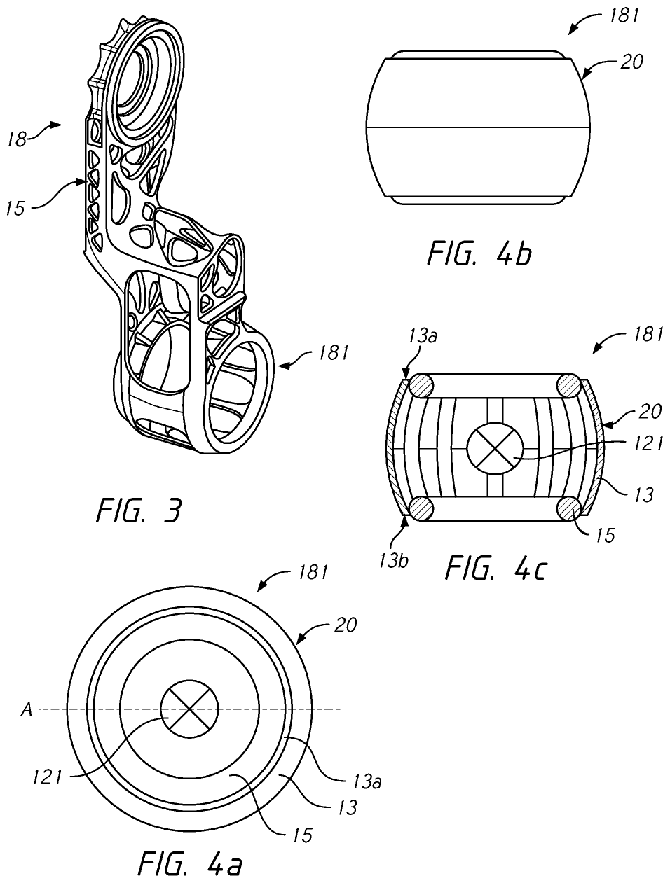

An example implementation of the proposed technique in a robot part of the robot 1, more specifically in a robot arm section, will now described with reference to FIG. 3 to FIG. 5.

In robotics, topologically optimized structures with load bearing structures having high strength, are generally desirable due to their light weight. Light weight is also a key requirement for robots placed on mobile robot platforms. Such topologic optimized structures are commonly obtained by additive manufacturing a.k.a. 3D printing. Typical materials used for the load bearing structure could be metallic materials (high strength steels, titanium, aluminum and their alloys) or composite materials. Hence, engineering high stiffness materials may be preferred.

In order to allow a maximum of areas where light can pass through, a topologic optimized structure using a very high specific strength might also be beneficial as it typically comprises a hollow structure with large openings for the light to pass through the structure without shadowing the inner light.

FIG. 3 illustrates an example of a load bearing structure, herein referred to as simply “a structure 15”, of the lower robot arm section 18. The lower robot arm section 18 comprises an arched cylinder-shaped joint part 181 that is designed to contain gear and motor of the corresponding joint 19. When mounted, the top base and the bottom base of the cylinder-shaped joint part 181 are movably connected to the next arm section, while the side of the cylinder-shaped joint part 181 defines a part of the exterior surface 20 of the robot 1.

FIGS. 4a to 4c illustrate a first example embodiment of the cylinder-shaped joint part 181. FIG. 4a illustrates the cylinder-shaped joint part 181 seen from above (i.e. from the top base), FIG. 4b illustrates the cylinder-shaped joint part 181 seen from the side and FIG. 4c illustrates a cross section of the cylinder-shaped joint part 181 along the dash dotted line A (FIG. 4a).

The cylinder-shaped joint part 181 comprises a part of the structure 15 and a casing 13 wrapped around that part of the structure 15. A light source 121 emitting disinfecting light, here embodied as a lamp, is arranged inside the structure 15. The casing 13 is a shell (having an upper edge 13a and a lower edge 13b) which is at least partly made from a material transparent to the disinfecting light. Hence, light emitted by the light source 121 can pass through the casing 13 and thereby illuminate the exterior surface 20 from the inside. In other words, in some embodiments the robot 1 comprises a hollow structure 15 and the at least one light emitting component 12 comprises a light source 121 arranged inside the hollow structure 15. In some embodiments, the exterior surface 20 is at least partly defined by a casing 13 and the light emitting component 12 is configured to emit the disinfecting light from a position inside the casing 13. In some embodiments, the casing 13 is made from a light diffusing material, whereby the disinfected light is evenly distributed at the exterior surface 20 defined by the casing 13. From the outside, the structure 15 may appear slightly darker than the rest of the cylinder-shaped joint part 181 due to shadowing effect.

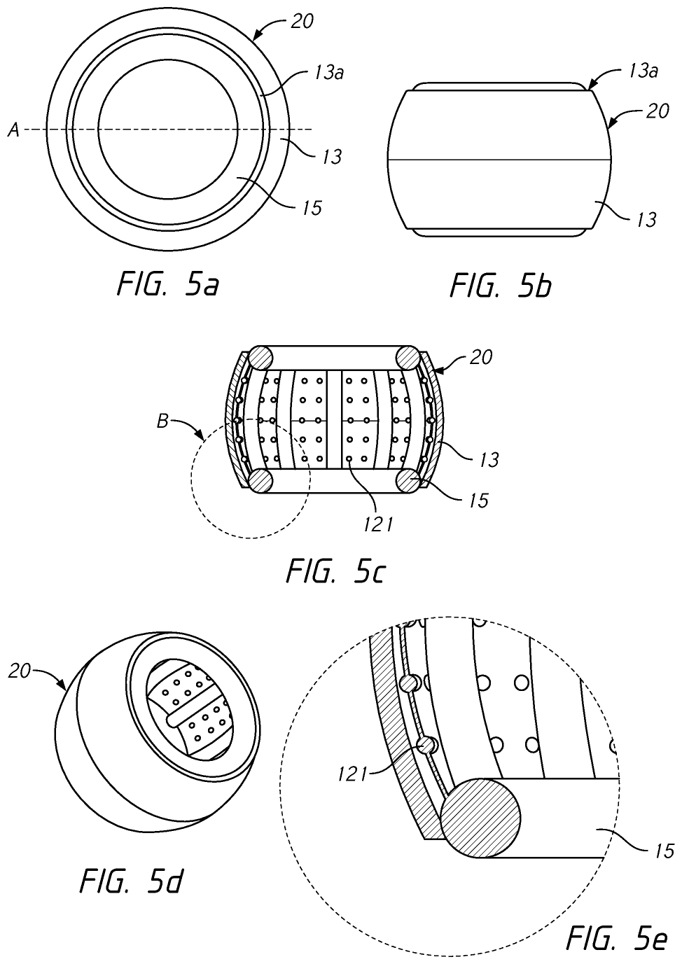

FIGS. 5a to 5e illustrate a second example embodiment of the cylinder-shaped joint part 181. FIG. 5a illustrates the cylinder-shaped joint part 181 seen from above (i.e. from the top base), FIG. 5b illustrates the cylinder-shaped joint part 181 seen from the side and FIG. 5c illustrates a cross section of the cylinder-shaped joint part 181 along the dash dotted line A (FIG. 5a). FIG. 5d illustrates the cylinder-shaped joint part 181 seen diagonally from above and FIG. 5e shows an enlargement of the section B in FIG. 5c, illustrating the placement of the light sources (here illustrated as diodes) 121 in further detail.

This embodiment differs from the first embodiment in that the light emitting component comprises a plurality of light sources 121, here illustrated as light diodes integrated in the casing 13. The diodes are arranged to illuminate the exterior surface 20 from inside the robot 1. In other words, in some embodiments, the light emitting component 12 is configured to emit the disinfecting light from a position inside the casing 13 or from a position integrated in the casing 13.

Another critical aspect for the use of service robots is the mobility. As mentioned above, placing a robot on a moving platform in a possibly contaminated environment might be foreseen as the fastest way to spread a contaminant through the entire factory. Here, again, continuous disinfection using disinfecting light might be a “sine qua non” for the application and a decisive asset compared to daily washdown solution. For example, a light emitting component 12 may be arranged to disinfect the surface under and around the robot 1. This is particularly relevant for a robot 1 positioned on a mobile platform, i.e. a platform that enables the robot 1 to move around during operation. In other words, in some embodiments, the at least one light emitting component 12 is configured to emit disinfecting light on the surface, on which the mobile platform is disposed, and/or on one or more transportation means of the mobile platform, to enable sanitization during movement of the mobile platform. To achieve this light emitting components are for example arranged to emit light diagonally downwards from the vertical sides of the mobile platform.

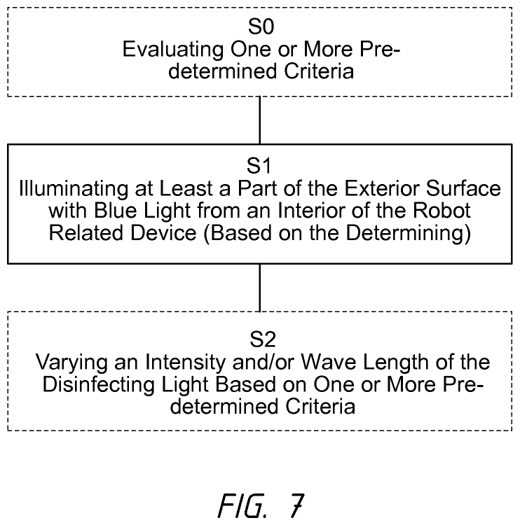

In some embodiments the robot 1 comprises, or is connected to, a control unit 50. The control unit 50 comprises a processor and a memory. The control unit 50 is for example an external computer, or a robot controller of the robot 1. The memory may include a computer program, wherein the computer program comprises a computer program code to cause the control unit 50, or a computer connected to the control unit 50, to control the at least one light emitting component and/or the optical component 14, 124 to illuminate at least a part of the exterior surface with disinfecting light from the inside of the robot. More specifically, the control unit 50 is configured to perform the method as will be described in the following. The program may be stored on a computer-readable medium, such as a memory stick or a CD ROM. A computer program product may comprise a computer program code stored on such a computer-readable medium to perform the method for disinfecting a robot as described in connection with FIG. 7, when the computer program code is executed by the control unit 50 or by a computer connected to the control unit 50.

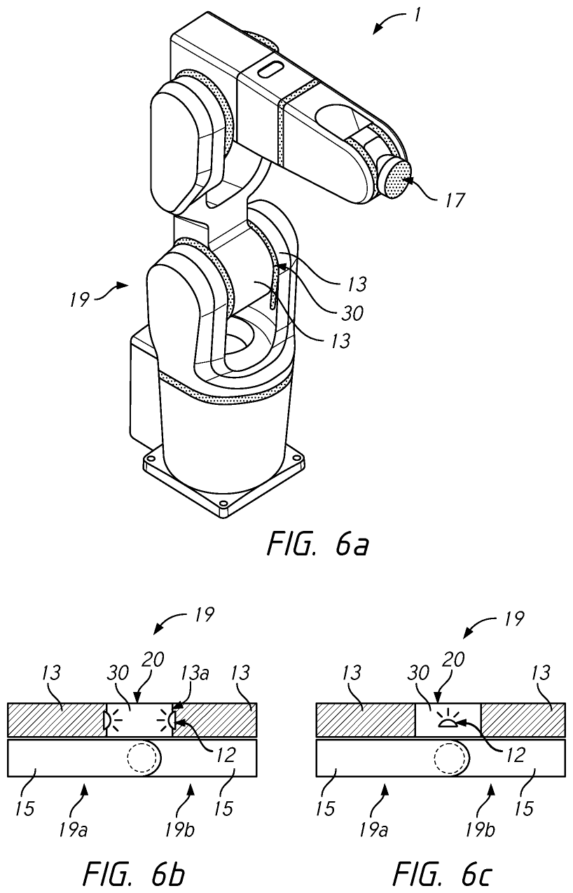

FIG. 6a illustrates another example embodiment of the self-disinfecting robot 1, where only the gripper and dynamic seals of the joints 19 of the robot 1 are disinfected. In this example, washdown areas, for example made of stainless steel, are not illuminated. Instead only dynamic seals that are especially difficult to reach by conventional washing are sanitised, because the seal will prevent washing of the joint faces if bacteria by accident enters the joint, which may happen at high load. The illumination might be done by using blue LED or another light source arranged in a ring or in a way to cover the contaminated areas of the gasket. The final design would then consist of a stainless-steel robot with blue glowing rings in the different axes or gaskets.

In other words, in this example, the at least one light emitting component 12 (FIG. 6b, FIG. 6c) is arranged to illuminate parts of the exterior surface 20 adjacent to one or more joints 19 and/or interfaces 17 of the robot 1. These areas are typically exposed as dirt and bacteria may enter the robot at the joints 19.

FIG. 6b is a conceptual illustration of a cross-section of a part of a joint 19. The joint typically comprises two parts 19a, 19b arranged to have a relative movement in between. Each part comprises a bearing structure 15 and casing 13. A gasket, herein referred to as sealing device 30, is typically arranged in a gap between the joint parts 19a, 19b, or more specifically between their casings 13 to prevent that fluid or other material can enter the joint 19 during working or wash down, and also avoid that fluid such as grease can come out of the joint 19 and potentially contaminate the food that is being processed.

In other words, the exterior surface 20 is in this example defined by both the casing 13 and the sealing device 30. The sealing device 30 is transparent to the disinfecting light. A light emitting component 12 is arranged at the edge 13a of the casing 13 that is facing the sealing device 30. Thereby, the gap between the parts 19a, 19b is disinfected. At least a part of the disinfecting light will then pass through the sealing device 30 and illuminate a part of the exterior surface 20 that is defined by the sealing device 30. In other words, the light emitting component 12 is arranged to illuminate the exterior surface 20 (laterally) from the side of the sealing device 3 (along the exterior surface 20) or from below the sealing device 30.

An alternative placement of the light emitting component 12 is illustrated in FIG. 6c. In this example the light emitting component 12 is integrated in the sealing device 30 and illuminates the part of the exterior surface 20 that is defined by the sealing device 30 from below. FIGS. 6a and 6b only exemplifies how the light emitting component 12 may be placed to illuminate the exterior surface 20 that is defined by the sealing device 30. The light emitting component 12 may alternatively be arranged in other ways, e.g. it may be only partly integrated in the sealing device 30.

FIG. 7 illustrates a method for disinfecting a robot 1 comprising an exterior surface 20. The method is either performed during normal operation of the robot 1 or during cleaning and/or service. The method is typically implemented in a control system of the robot, such as in a robot controller 50 (FIG. 1).

The steps of the method may be defined in a computer program, comprising instructions which, when the program is executed by processors (e.g. processor of the control unit 50), causes the control unit 50 to carry out the method. The steps of the method may also be defined in a computer-readable medium, e.g. an internal memory of the control unit 50 and/or in any other memory. The computer-readable medium comprises instructions that, when executed by a control unit 50, causes the control unit 50 to carry out the method.

The method comprises illuminating S1 at least a part of the exterior surface 20 with disinfecting light from an interior of the robot 1. The illuminating may be continuous, pulsed or only triggered based on one or more pre-determined criteria. The illuminating S1 may e.g. be adapted depending on the degree of contamination. For example, the disinfecting light may be flashing, stroboscopic to achieve optimal disinfection for different circumstances. In some embodiments, the illuminating is triggered only at given times of the day, e.g. during night.

In some embodiments, the method comprises varying S2 an intensity and/or wavelength of the disinfecting light based on one or more pre-determined criteria. For example, the pre-determined criteria take input from sensors that monitor the type and degree of contamination. The pre-determined criteria may also include a schedule, such that the illumination is altered at different times of the day/night. The intensity and wavelength may e.g. be varied based on material, presence of people, degree of contamination etc. For example, UV light with better disinfecting effect may be used at night, when no people are present.

The present invention is not limited to the above-described preferred embodiments. Various alternatives, modifications and equivalents may be used. Therefore, the above embodiments should not be taken as limiting the scope of the invention, which is defined by the appending claims.

1. A self-disinfecting robot comprising an exterior surface, wherein the self-disinfecting robot comprises:
at least one light emitting component arranged to illuminate at least a part of the exterior surface with disinfecting light from inside the self-disinfecting robot.

2. The self-disinfecting robot according to claim 1, wherein the at least one light emitting component is configured to illuminate the exterior surface from the inside by emitting the disinfecting light from below the exterior surface or from the side along the exterior surface.

3. The self-disinfecting robot according to claim 1, wherein the at least one light emitting component comprises:
an internal light source configured to emit the disinfecting light arranged inside the self-disinfecting robot and/or
a light guide including at least one aperture, wherein the light guide is arranged to guide the disinfecting light from an external light source, through the light guide and out through the at least one aperture.

4. The self-disinfecting robot according to claim 1, wherein the self-disinfecting robot is designed such that at least a part of the disinfecting light can pass through, at least parts of, the exterior surface.

5. The self-disinfecting robot according to claim 1, wherein the at least one light emitting component is arranged to illuminate parts of the exterior surface adjacent to one or more joints and/or interfaces of the self-disinfecting robot.

6. The self-disinfecting robot according to claim 1, wherein the exterior surface is at least partly defined by a casing and wherein the light emitting component is configured to emit the disinfecting light from a position inside the casing or from a position integrated in the casing.

7. The self-disinfecting robot according to claim 1, comprising:
an optical component configured to spread the disinfecting light along the exterior surface, to guide the light to the surface and/or to focus the light on parts of the exterior surface.

8. The self-disinfecting robot according to claim 1, wherein the optical component comprises a light guide surface, a light diffusing material and/or a lens.

9. The self-disinfecting robot according to claim 1, wherein a part of the exterior surface is defined by a sealing device and the at least one light emitting component is at least partly integrated in the sealing device or the at least one light emitting component is arranged to illuminate the exterior surface from the side of the sealing device along the exterior surface or from below the sealing device.

10. The self-disinfecting robot according to claim 1, wherein the disinfecting light has a wavelength of 200-500 nm.

11. The self-disinfecting robot according to claim 1, wherein the exterior surface is at least partly covered by a contaminant-activated photocatalytic polymer or glass.

12. The self-disinfecting robot according to claim 1, wherein the robot is mounted on a mobile platform and wherein the at least one light emitting component is configured to emit disinfecting light on the surface on which the mobile platform is disposed, and/or on one or more transportation means of the mobile platform, to enable sanitization during movement of the mobile platform.

13. A method for disinfecting a self-disinfecting robot including an exterior surface, the method comprising:
illuminating at least a part of the exterior surface with disinfecting light from an interior of the self-disinfecting robot.

14. The method according to claim 13, wherein the illuminating is continuous, pulsed or triggered based on one or more pre-determined criteria.

15. The method according to claim 13, comprising varying an intensity and/or wavelength of the disinfecting light based on one or more pre-determined criteria.

16. The self-disinfecting robot according to claim 2, wherein the at least one light emitting component comprises:
an internal light source configured to emit the disinfecting light arranged inside the self-disinfecting robot and/or
a light guide including at least one aperture, wherein the light guide is arranged to guide the disinfecting light from an external light source, through the light guide and out through the at least one aperture.

17. The self-disinfecting robot according to claim 2, wherein the self-disinfecting robot is designed such that at least a part of the disinfecting light can pass through, at least parts of, the exterior surface.

18. The self-disinfecting robot according to claim 2, wherein the at least one light emitting component is arranged to illuminate parts of the exterior surface adjacent to one or more joints and/or interfaces of the self-disinfecting robot.

19. The self-disinfecting robot according to claim 2, wherein the exterior surface is at least partly defined by a casing and wherein the light emitting component is configured to emit the disinfecting light from a position inside the casing or from a position integrated in the casing.
